(19) United States
(12) Patent Application Publication
(10) Pub. No.: US 2026/0094721 A1
(43) Pub. Date: Apr. 2, 2026

(54) TRAINING DEVICE, TRAINING METHOD, DISEASE RISK ESTIMATION DEVICE, DISEASE RISK ESTIMATION METHOD, AND PROGRAM

(71) Applicant: NEC Corporation, Tokyo (JP)

(72) Inventors: Chenhui HUANG, Tokyo (JP); Kensuke WAGATA, Tokyo (JP); Fumiyuki NIHEY, Tokyo (JP)

(73) Assignee: NEC Corporation, Tokyo (JP)

(21) Appl. No.: 19/332,348

(22) Filed: Sep. 18, 2025

(30) Foreign Application Priority Data
Oct. 1, 2024 (JP) 2024-171955

(51) Int. Cl. G16H 50/30; G16H 50/20; G16H 50/70

(52) U.S. Cl. CPC G16H 50/30; G16H 50/20; G16H 50/70

(57) ABSTRACT

In order to estimate a future disease risk based on current data, a disease risk estimation device estimates a disease risk using AI or a machine learning model. An acquisition means acquires a current age, a future age, and current attribute data other than the age. An encoder projects the attribute data to a latent space according to a category of the age and clusters obtained projection points into a plurality of clusters. A predictor predicts disease risks based on positions of the projection points on the latent space. An output means outputs a prediction result of the disease risk. The prediction result of the disease risk is used to support decision making related to an activity of a subject.

### INCORPORATION BY REFERENCE

This application is based upon and claims the benefit of priority from Japanese Patent Application 2024-171955, filed on Oct. 1, 2024, the disclosure of which is incorporated herein in its entirety by reference.

### TECHNICAL FIELD

This disclosure relates to estimation of a disease risk.

### BACKGROUND ART

A disease risk estimation technology using a machine learning model is known. For example, Patent Document 1 describes a method of classifying data related to health into a high incidence risk group and a low incidence risk group and evaluating disease risks.
Patent Document 1: Japanese Patent Application Laid-Open under No. 2022-182943

### SUMMARY

In a method of Patent Document 1, a current disease risk can be estimated, but a future disease risk cannot be estimated.

One object of the present disclosure is to provide a disease risk estimation device capable of estimating a future disease risk based on current data.

According to an example aspect of the present invention, there is provided a training device comprising:
at least one first memory configured to store instructions; and
at least one first processor configured to execute the instructions to:
acquire an age and attribute data other than the age;
project, by an encoder, the attribute data to a latent space according to a category of the age and cluster obtained projection points into a plurality of clusters;
predict, by a predictor, disease risks based on positions of the projection points on the latent space; and
optimizes the encoder and the predictor based on relationships between the projection points in the latent space and the plurality of clusters and a mutual relationship between the plurality of clusters.

According to another example aspect of the present invention, there is provided a training method executed by a computer, the training method comprising:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

According to still another example aspect of the present invention, there is provided a recording medium recording a program, the program causing a computer to execute processing of:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

According to a further example aspect of the present invention, there is provided a disease risk estimation device comprising:
at least one second memory configured to store instructions; and
at least one second processor configured to execute the instructions to:
acquire a current age, a future age, and current attribute data other than the age;
project, by an encoder, the attribute data to a latent space according to a category of the age and clusters obtained projection points into a plurality of clusters;
move, in the latent space, a projection point related to the current age to a position related to the future age;
predict, by a predictor, a disease risk based on the position of the projection point on the latent space; and
output a prediction result of the disease risk.

According to a still further example aspect of the present invention, there is provided disease risk estimation method executed by a computer, the disease risk estimation method comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.

According to a yet still another example aspect of the present invention, there is provided a non-transitory computer-readable recording medium storing a program for causing a computer to execute processing comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.

### Effect

According to the present disclosure, it is possible to estimate a future disease risk based on current data.

### BRIEF DESCRIPTION OF THE DRAWINGS

FIG. 1 illustrates an overall configuration of a disease risk estimation device according to the present disclosure;

FIG. 2 is a block diagram illustrating a hardware configuration of the disease risk estimation device;

FIG. 3 is a block diagram illustrating a functional configuration of a training device of a disease risk estimation model;

FIG. 4 schematically illustrates a latent space;

FIG. 5 is a flowchart of training processing;

FIG. 6 is a block diagram illustrating a functional configuration of the disease risk estimation device;

FIG. 7 schematically illustrates the latent space;

FIG. 8 is a flowchart of disease risk estimation processing;

FIG. 9 is a block diagram illustrating a functional configuration of a disease risk estimation device according to a modification;

FIG. 10 schematically illustrates a latent space in the modification;

FIG. 11 is a block diagram illustrating a functional configuration of a training device of a second example embodiment;

FIG. 12 is a flowchart of processing by the training device of the second example embodiment;

FIG. 13 is a block diagram illustrating a functional configuration of a disease risk estimation device of a third example embodiment; and

FIG. 14 is a flowchart of processing by the disease risk estimation device of the third example embodiment.

### EXAMPLE EMBODIMENTS

Hereinafter, preferred example embodiments of the present disclosure will be described with reference to the drawings.

### First Example Embodiment

### [Overall Configuration]

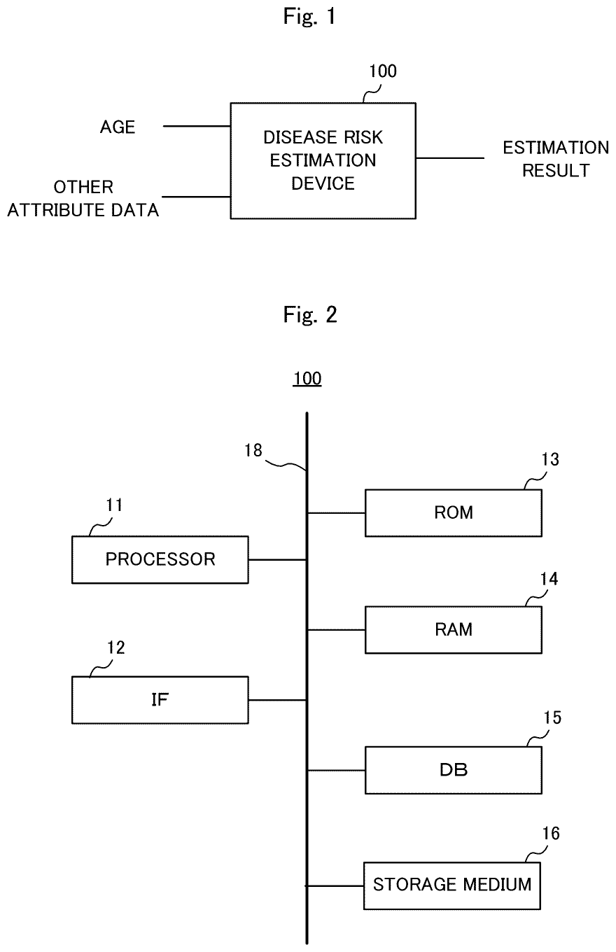

FIG. 1 illustrates an overall configuration of a disease risk estimation device according to a first example embodiment of the present disclosure. A disease risk estimation device 100 estimates a disease risk of a subject based on data related to health of the subject. Specifically, an age and attribute data other than the age of the subject are input to the disease risk estimation device 100. The disease risk estimation device 100 estimates the disease risk of the subject by using a disease risk estimation model based on the age and the other attribute data, and outputs an estimation result. The disease risk estimation model is an artificial intelligence (AI) or machine learning model trained by a training phase to be described later. The disease risk estimation device 100 of the present disclosure can predict not only a current disease risk but also a future disease risk by using a probability distribution feature of data of each age group based on the age.

The disease risk estimation device 100 can be suitably applied to a medical or healthcare field. For example, the disease risk estimation device 100 can be used when a risk of a lifestyle disease is estimated based on data obtained in a periodic medical examination.

### [Hardware Configuration]

FIG. 2 is a block diagram illustrating a hardware configuration of the disease risk estimation device 100. As illustrated in the drawing, the disease risk estimation device 100 includes a processor 11, an interface (IF) 12, a read only memory (ROM) 13, a random access memory (RAM) 14, a database (DB) 15, and a storage medium 16. The components are connected to each other via, for example, a bus 18.

The processor 11 is a computer such as a central processing unit (CPU), and controls the entire disease risk estimation device 100 by executing a program prepared in advance. Specifically, as the processor 11, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these can be used.

The processor 11 loads a program stored in the ROM 13 or the storage medium 16 into the RAM 14, and executes each type of processing coded in the program. The processor 11 functions as a part or all of the disease risk estimation device 100. Specifically, the processor 11 executes training processing and disease risk estimation processing to be described later.

The IF 12 transmits and receives data to and from an external device. Specifically, in the training phase, the disease risk estimation device 100 receives an age, other attribute data, a true value of a disease risk, and the like as training data via the IF 12. In an estimation phase, that is, at the time of estimation of a disease risk, via the IF 12, the disease risk estimation device 100 receives an age and other attribute data of a subject, and outputs an estimation result of the disease risk to a display device or another external device.

The ROM 13 stores various programs executed by the processor 11. The RAM 14 is used as a working memory during execution of various types of processing by the processor 11.

The DB 15 stores various algorithms, data, a machine learning model, and the like used when the disease risk estimation device 100 executes the training processing and the disease risk estimation processing to be described later.

The storage medium 16 is a non-volatile non-transitory storage medium such as a disk-shaped recording medium or a semiconductor memory. The storage medium 16 may be attachable to and detachable from the disease risk estimation device 100. The storage medium 16 records various programs executed by the processor 11.

In addition to the above, the disease risk estimation device 100 may include a display device such as a liquid crystal display and an input device such as a keyboard and a mouse. The display device and the input device are used by, for example, an operator of the disease risk estimation device 100.

### [Training Phase]

Next, the training phase of the disease risk estimation model will be described.

### (Training Device)

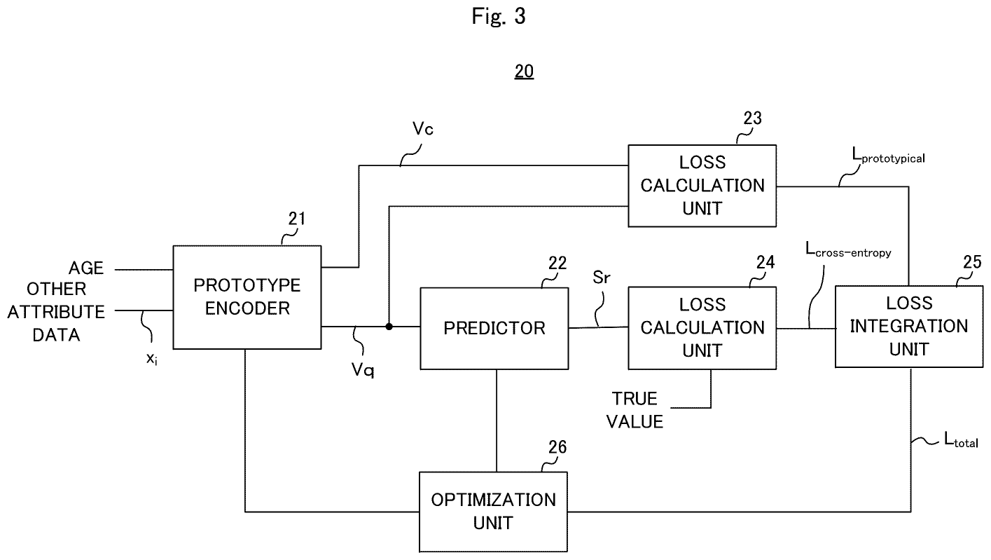

As described above, the disease risk estimation device 100 estimates the disease risk by using the trained disease risk estimation model. FIG. 3 is a block diagram illustrating a functional configuration of a training device 20 of the disease risk estimation model. The training device 20 trains the disease risk estimation model by prototype training. As illustrated in the drawing, the training device 20 includes a prototype encoder 21, a predictor 22, loss calculation units 23 and 24, a loss integration unit 25, and an optimization unit 26.

The disease risk estimation model includes a pair of the prototype encoder 21 and the predictor 22. Specifically, the prototype encoder 21 and the predictor 22 are configured by a neural network. In the training phase, the training device 20 generates the trained disease risk estimation model by optimizing the neural network by using training data.

As the training data, disease risk data related to a plurality of persons is prepared. Specifically, the training data is data obtained by collecting, for each of the plurality of persons, an age, other attribute data, and a disease risk value of the person. As the other attribute data, for example, data having high relevance with the disease risk to be estimated is used among a height, a weight, a gender, a body mass index (BMI), presence or absence and amount of smoking, presence or absence and amount of drinking, and the like. The disease risk value of the person relates to correct answer data in so-called supervised learning, and is hereinafter also referred to as a “true value”.

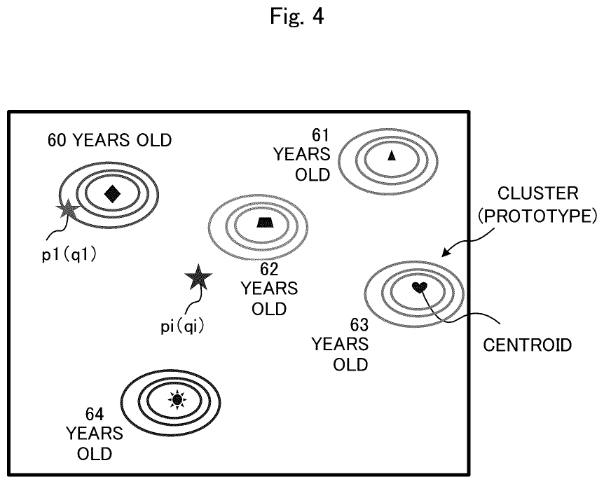

In FIG. 3, first, an age and other attribute data xi other than the age are input to the prototype encoder 21 (hereinafter also simply referred to as the “encoder 21”). The encoder 21 projects the input attribute data xi to a latent space. FIG. 4 schematically illustrates the latent space. The “latent space” is an abstract space for expressing information included in original data in fewer dimensions, and in the latent space, essential features and patterns of the data are expressed in the fewer dimensions. “Projects . . . to a latent space” refers to converting the original data into points on the latent space, which is also referred to as “maps . . . to a latent space”. Hereinafter, points on a latent space obtained by projecting certain data to the latent space are also referred to as “projection points”.

The encoder 21 projects the attribute data of the plurality of persons included in the training data to the latent space. As a result, a large number of the projection points are mapped onto the latent space. In FIG. 4, a position of the projection point in the latent space is represented by “p”, and a feature amount (also referred to as a “latent vector”, a “feature vector”, or simply a “vector” or the like) related to the position is represented by “q”. In the example of FIG. 4, it is indicated that certain attribute data dl is projected to a projection point p1 and a feature amount related to the projection point p1 is q1. It is also indicated that certain attribute data di is projected to a projection point pi and a feature amount related to the projection point pi is qi.

The encoder 21 projects the plurality of pieces of attribute data included in the training data to the latent space according to the age, and clusters the obtained projection points. Specifically, the encoder 21 clusters the projection points for each category of the age, and generates a cluster for each category of the age. The category of the age can be optionally set, and may be, for example, a category for every one year of age, or a category for every five years of age. In the example of FIG. 4, the category of the age is set for every one year of age, and the encoder 21 generates clusters “60 years old”, “61 years old”, . . . for each age. These clusters are also referred to as “prototypes”, and a center of gravity of each cluster (prototype) is referred to as a “centroid”. In this manner, the encoder 21 can generate the cluster according to the category of the age by using the input age.

After clustering the plurality of projection points, the encoder 21 outputs a feature amount (hereinafter referred to as a “centroid vector”) Vc of the center of gravity of each cluster. The centroid vector Vc is represented by the following expression.

$$Vc = [ μ1 , … , μ i , … , μ C ] \qquad ( 1 )$$

Note that the centroid vector of each cluster is indicated by “u”, and the number of clusters is indicated by “C”.

The encoder 21 also outputs a feature amount (hereinafter referred to as a “projection point vector”) Vq of each projection point to the predictor 22 and the loss calculation unit 23. The projection point vector Vq is represented as follows. The number of projection points is indicated by “N”.

$$Vq = [ q 1 , … , qi , … , qN ] \qquad ( 2 )$$

The predictor 22 calculates a score of the disease risk (hereinafter referred to as a “risk score”) Sr related to each piece of the attribute data based on an input projection point vector Vq, and outputs the risk score Sr to the loss calculation unit 24.

The loss calculation unit 23 calculates a first loss L_{prototypical} by the following Expression (3) by using the input centroid vector Vc and projection point vector Vq, and outputs the first loss L_{prototypical} to the loss integration unit 25.

$$\begin{matrix}[ Math 1 ] & \\ {ℒ}_{prototypical} = {\underbrace{- \frac{1}{N} {{\sum }^{N}}_{i = 1} log ( \frac{exp ( - d ( {q}_{i} , {μ}_{i} ) )}{{\sum }_{j = 1}^{C} exp ( - d ( {q}_{i} , {μ}_{j} ) )} )}}_{First term} + {\underbrace{λ {{\sum }^{C}}_{j = 1} {{\sum }^{C}}_{k = 1 , k \neq j} \frac{1}{d ( {μ}_{j} , {μ}_{k} )}}}_{Second term} & ( 3 )\end{matrix}$$

In Expression (3), a function d(q, u) indicates a distance between the projection point vector q and the centroid vector u. Therefore, a denominator in parentheses in a first term of Expression (3) indicates a sum of distances between a certain projection point and centroids of clusters. A numerator in the parentheses in the first term indicates a distance between the projection point and a centroid of a cluster to which the projection point belongs. Therefore, the first term has a smaller value as the projection point belonging to the certain cluster is closer to the centroid of the cluster. On the other hand, a second term of Expression (3) indicates a sum of reciprocals of distances between the individual centroids. Therefore, the second term has a smaller value as the individual centroids are farther from each other. Therefore, the first loss L_{prototypical} decreases as a projection point belonging to a certain cluster is closer to a centroid of the cluster, and decreases as the individual centroids are farther from each other. Therefore, by using the first loss L_{prototypical}, the training device 20 performs training in such a way that a projection point in a cluster is close to a centroid of the cluster and centroids of clusters are far from each other in the latent space.

On the other hand, the loss calculation unit 24 calculates a cross entropy between the input risk score Sr of each piece of the attribute data and a true value related to the attribute data, and outputs a second loss L_{cross-entropy} to the loss integration unit 25.

The loss integration unit 25 calculates a weighted sum of the input first loss L_{prototypical} and second loss L_{cross-entropy} by the following Expression (4), and outputs the weighted sum to the optimization unit 26 as a total loss Lₜₒₜₐₗ.

$$\begin{matrix}[ Math 2 ] & \\ {ℒ}_{total} = {ℒ}_{prototypical} + {λℒ}_{cross - entropy} & ( 4 )\end{matrix}$$

Note that a weight when weighted addition of the first loss and the second loss is performed is indicated by “2”.

The optimization unit 26 optimizes the encoder 21 and the predictor 22 based on the total loss Lₜₒₜₐₗ. Specifically, the optimization unit 26 optimizes parameters of the neural network constituting the encoder 21 and the predictor 22 in such a way that the total loss Lₜₒₜₐₗ becomes small. Here, as described above, since the total loss Lₜₒₜₐₗ is the weighted sum of the first loss L_{prototypical} and the second loss L_{cross-entropy}, the optimization unit 26 performs the optimization in such a way that, in the latent space, (A) a projection point in a cluster is close to a centroid of the cluster, (B) centroids of clusters are far from each other, and (C) the risk score predicted by the predictor 22 for the attribute data included in the training data is close to the true value.

In this manner, the training device 20 generates the disease risk estimation model that estimates the disease risk related to the input attribute data in relation to the cluster for each age obtained on the latent space.

### (Training Processing)

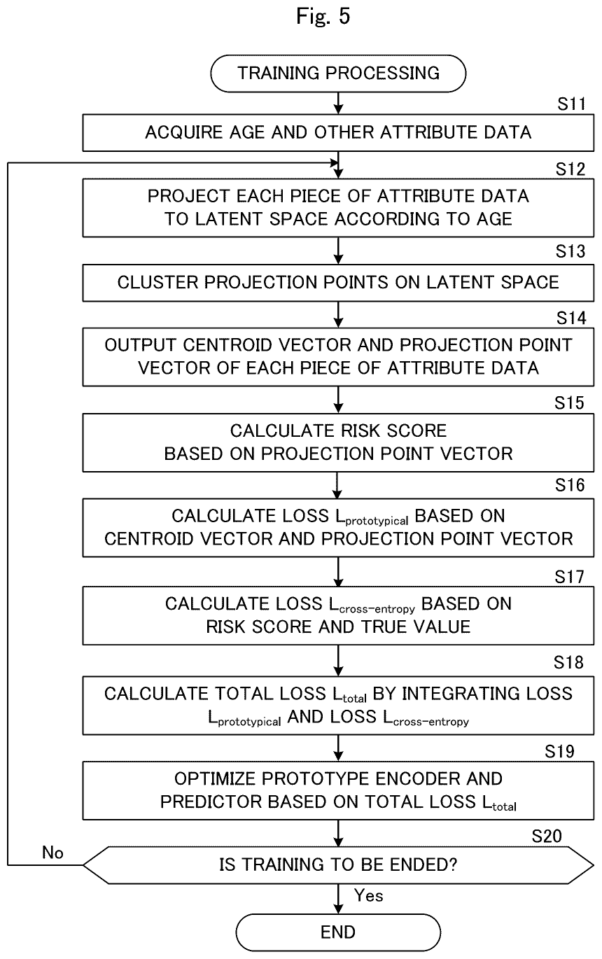

Next, the training processing executed by the above training device 20 will be described. FIG. 5 is a flowchart of the training processing. This processing is achieved by the processor 11 illustrated in FIG. 2 executing a program prepared in advance and operating as the components illustrated in FIG. 3.

First, the encoder 21 acquires the age and the other attribute data included in the training data (step S11). Next, the encoder 21 projects each piece of the attribute data to the latent space according to the age (step S12), and clusters the projection points on the latent space (step S13). The encoder 21 then outputs the centroid vector of each cluster and the projection point vector of each projection point to the predictor 22 (step S14). Next, the predictor 22 calculates the risk score related to each piece of the attribute data based on the centroid vector and the projection point vector (step S15).

Next, the loss calculation unit 23 calculates the first loss L_{prototypical} based on the centroid vector and the projection point vector (step S16). The loss calculation unit 24 also calculates the second loss L_{cross-entropy} based on the risk score and the true value (step S17). Next, the loss integration unit 25 calculates the total loss Lₜₒₜₐₗ by integrating the first loss L_{prototypical} and the second loss L_{cross-entropy} (step S18). The optimization unit 26 then optimizes the encoder 21 and the predictor 22 based on the total loss Lₜₒₜₐₗ (step S19).

Next, the training device 20 determines whether a predetermined training end condition is satisfied (step S20). Examples of the training end condition include that a predetermined number of pieces of the attribute data prepared as the training data is used, the total loss has become equal to or less than a predetermined value, and the total loss has converged. In a case where the training end condition is not satisfied (step S20: No), the processing returns to step S12. On the other hand, in a case where the training end condition is satisfied (step S20: Yes), the training processing ends.

### [Estimation Phase]

Next, the estimation phase of the disease risk estimation device will be described. In the estimation phase, the disease risk estimation device 100 estimates current and future disease risks of a certain subject based on attribute data of the subject. At this time, the disease risk estimation device 100 uses the disease risk estimation model trained in the training phase, specifically, the encoder 21 and the predictor 22.

### (Disease Risk Estimation Device)

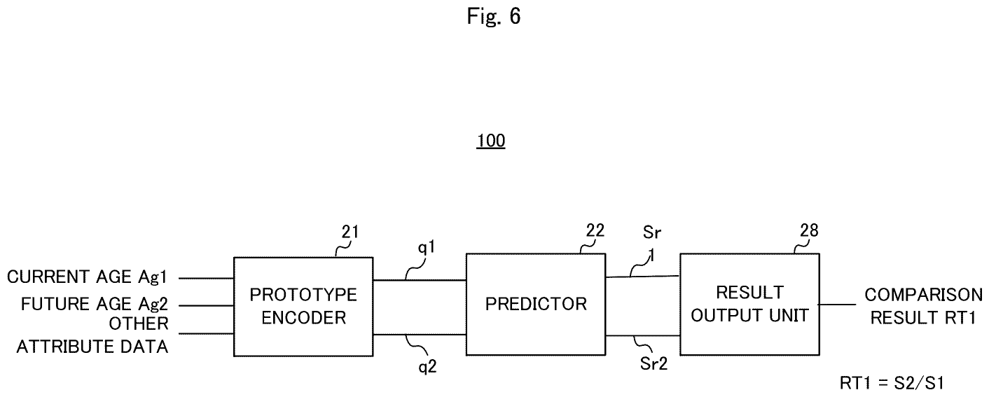

FIG. 6 is a block diagram illustrating a functional configuration of the disease risk estimation device. The disease risk estimation device 100 includes the encoder 21 and the predictor 22 optimized in the training phase, and a result output unit 28.

A current age Ag1, a future age Ag2, and other attribute data other than the age are input to the encoder 21 for a certain subject. The future age Ag2 is an age at which the subject desires to know a disease risk, that is, an age to be estimated. In the following example, it is assumed that the current age of the subject is 60 years old, and a disease risk at 61 years old after one year is estimated. In this case, the future age Ag2 may be input as “61 years old” or “after one year”.

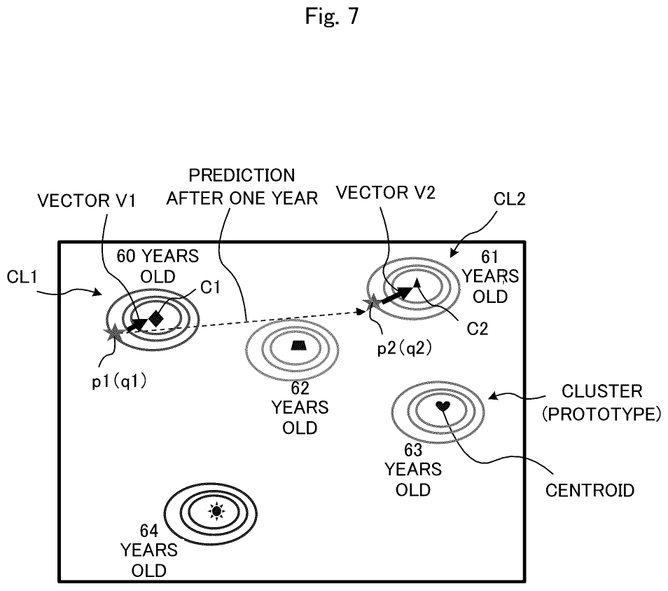

As illustrated in FIG. 7, the encoder 21 optimized in the training phase projects the input attribute data to the latent space including the cluster for each age category. In this example, first, the encoder 21 projects the attribute data related to the current age Ag1 to the projection point p1. A projection point vector of the projection point p1 is set as q1.

Next, the encoder 21 generates a projection point p2 related to the future age Ag2, that is, 61 years old, based on the projection point p1 related to the current age Ag1. Specifically, the encoder 21 moves the projection point p1 in a cluster CL1 of 60 years old related to the current age Ag1 to a cluster CL2 of 61 years old related to the future age Ag2, and sets the projection point p1 as the projection point p2 related to the future age Ag2. At this time, the encoder 21 generates the projection point p2 in such a way that a positional relationship between the projection point p1 and a centroid C1 in the cluster CL1 of 60 years old matches a positional relationship between the projection point p2 and a centroid C2 in the cluster CL2 of 61 years old after the movement. In other words, the encoder 21 generates the projection point p2 in such a way that a vector V1 from the projection point p1 toward the centroid C1 in the cluster CL1 of 60 years old matches a vector V2 from the projection point p2 toward the centroid C2 in the cluster CL2 of 61 years old. As a result, the projection point p2 becomes a projection point indicating the feature amount in a case where the other attribute data does not change and only the age changes to 61 years old for the subject. A projection point vector of the projection point p2 is set as q2.

In this manner, after generating the projection point p2 related to the future age Ag2, the encoder 21 outputs the projection point vector q1 of the projection point p1 and the projection point vector q2 of the projection point p2 to the predictor 22.

The predictor 22 predicts a risk score Sr1 at the current age Ag1 based on the projection point vector q1. The predictor 22 also predicts a risk score Sr2 at the future age Ag2 based on the projection point vector q2. The predictor 22 then outputs the risk scores Sr1 and Sr2 to the result output unit 28.

The result output unit 28 outputs a comparison result between the risk score Sr1 at the current age Ag1 and the risk score Sr2 at the future age Ag2. For example, the result output unit 28 calculates a ratio of the risk score Sr1 to the risk score Sr2:RT1=Sr2/Sr1, and outputs a message such as “The risk after one year is RT1 times the current risk.” In this manner, the subject can know how the future disease risk changes compared to the current disease risk.

### (Disease Risk Estimation Processing)

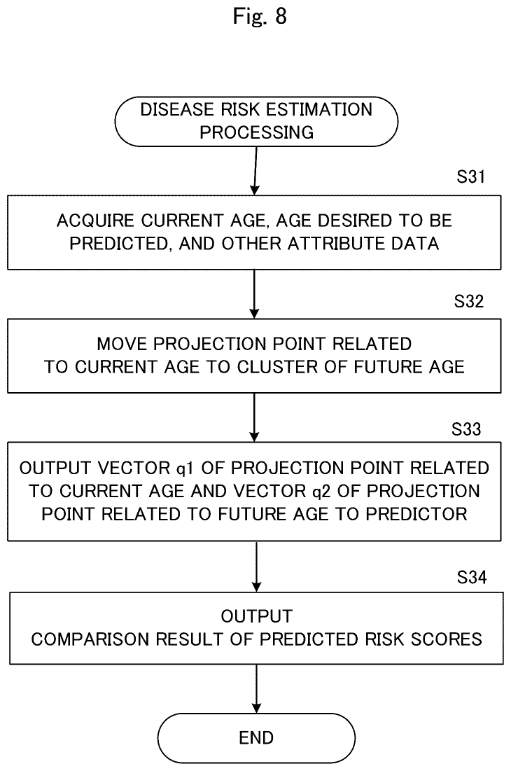

Next, the disease risk estimation processing executed by the above disease risk estimation device 100 will be described. FIG. 8 is a flowchart of the disease risk estimation processing. This processing is achieved by the processor 11 illustrated in FIG. 2 executing a program prepared in advance and operating as the components illustrated in FIG. 6.

First, the encoder 21 acquires the age, the future age, and the other attribute data for the subject of the risk estimation (step S31). Next, the encoder 21 moves the projection point related to the current age to the cluster related to the future age, and generates the projection point related to the future age (step S32). Next, the encoder 21 outputs the projection point vector q1 of the projection point p1 related to the current age and the projection point vector q2 of the projection point p2 related to the future age to the predictor 22 (step S33).

The predictor 22 calculates the risk scores Sr1 and Sr2 based on the projection point vectors q1 and q2, and outputs the comparison result of these (step S34). In this manner, the comparison result of the risk scores related to the current age and the future age is output. The disease risk estimation processing then ends.

### (Modification)

In the above disease risk estimation processing, the comparison result between the current risk score and the future risk score is output for the certain subject. Instead, a result obtained by comparing the risk score of the certain subject with an average value of risk scores of a large number of other persons, that is, a general risk score may be output.

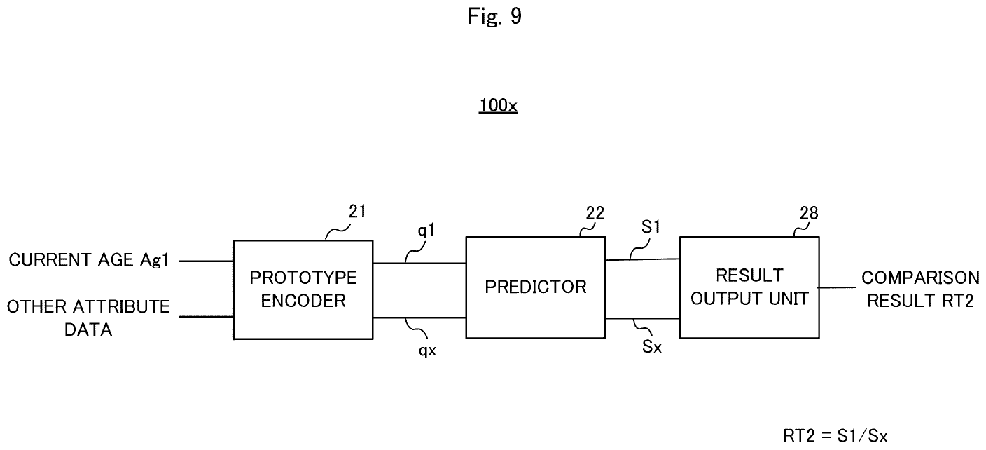

FIG. 9 is a block diagram illustrating a functional configuration of a disease risk estimation device 100x according to a modification. The disease risk estimation device 100x according to the modification has the configuration similar to that of the disease risk estimation device 100 illustrated in FIG. 6. However, the modification is different in that the current age Ag1 of the subject and other attribute data of a plurality of other persons are input to the encoder 21.

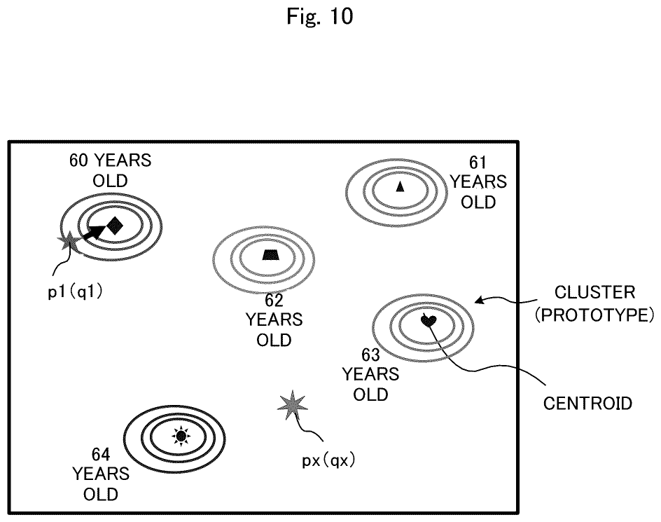

FIG. 10 schematically illustrates a latent space in the modification. The encoder 21 projects the current age Ag1 of the subject to the projection point p1 on the latent space, acquires the projection point vector q1, and outputs the projection point vector q1 to the predictor 22. The encoder 21 projects the attribute data of the plurality of other persons onto the latent space, and determines a projection point (hereinafter also referred to as an “average point”) px related to an average value of a plurality of obtained projection points as illustrated in FIG. 10. The encoder 21 then acquires an average point vector qx related to the average point, and outputs the average point vector qx to the predictor 22.

The predictor 22 predicts the risk score Sr1 at the current age Ag1 based on the projection point vector q1, and predicts an average risk score Sx based on the average point vector qx. The predictor 22 then outputs the risk score Sr1 and the average risk score Sx to the result output unit 28.

The result output unit 28 outputs a comparison result between the risk score Sr1 related to the current age Ag1 of the subject and the average risk score Sx. For example, the result output unit 28 calculates a ratio of the risk score Sr1 of the subject to the average risk score Sx:RT2=Sr1/Sx, and outputs a message such as “Your current risk is RT2 times the risk of a general adult.”. In this manner, the subject can know his/her own disease risk compared to a general person.

### [Modification]

In the above first example embodiment, an attribute data generation device is applied to generation of attribute data related to health of a person, but application of the present disclosure is not limited to this. For example, the present disclosure can also be applied to generation of attribute data detected and collected in inspection and diagnosis of a machine or a device.

### Second Example Embodiment

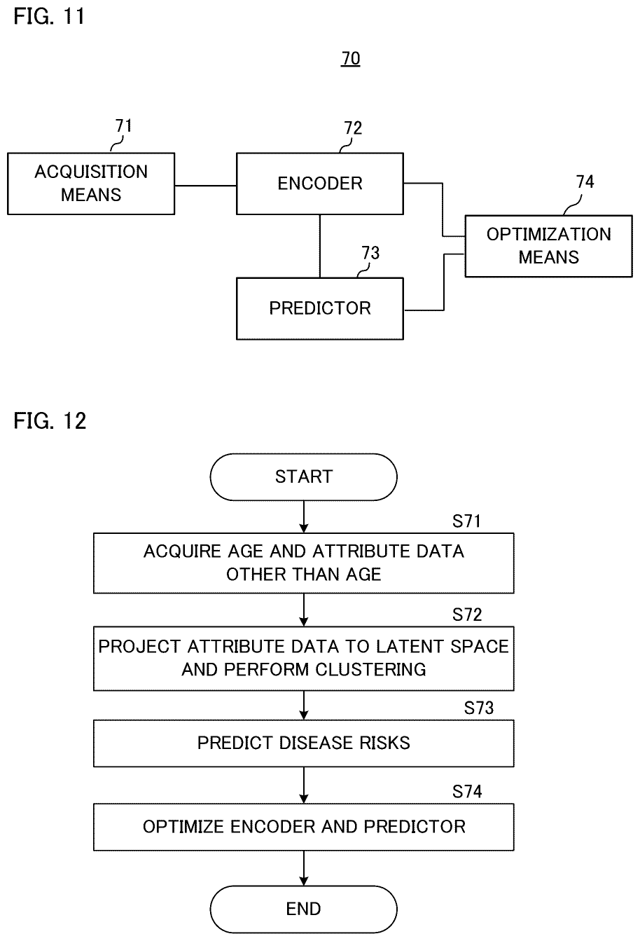

FIG. 11 is a block diagram illustrating a functional configuration of a training device of a second example embodiment. A training device 70 includes acquisition means 71, an encoder 72, a predictor 73, and optimization means 74.

FIG. 12 is a flowchart of processing by the training device of the second example embodiment. The acquisition means 71 acquires an age and attribute data other than the age (step S71). The encoder 72 projects the attribute data to a latent space according to a category of the age, and clusters obtained projection points into a plurality of clusters (step S72). The predictor 73 predicts disease risks based on positions of the projection points on the latent space (step S73). The optimization means 74 optimizes the encoder and the predictor based on relationships between the projection points and the plurality of clusters in the latent space and a mutual relationship between the plurality of clusters (step S74).

According to the training device 70 of the second example embodiment, a future disease risk can be estimated based on current data by the encoder and the predictor optimized by training.

### Third Example Embodiment

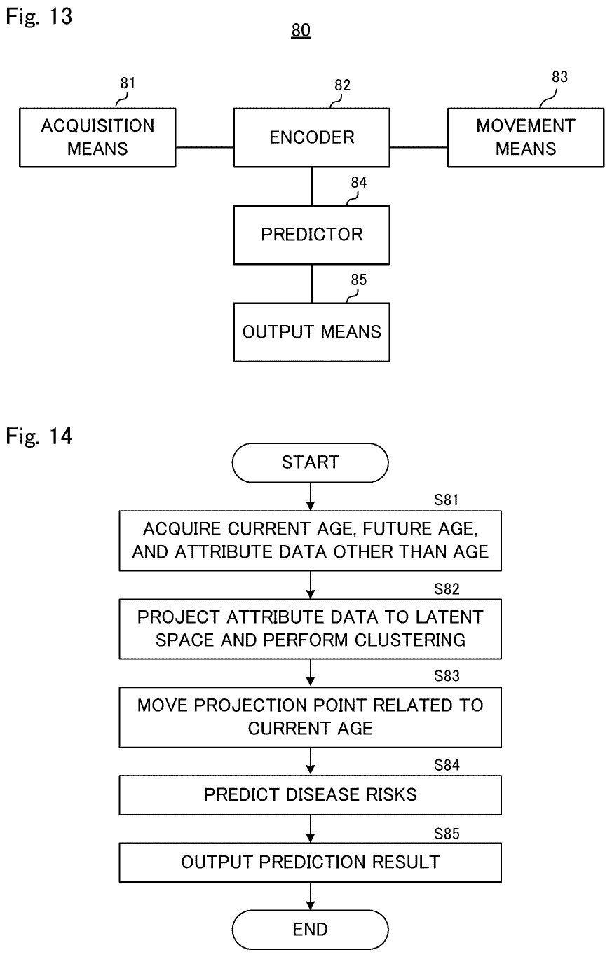

FIG. 13 is a block diagram illustrating a functional configuration of a disease risk estimation device of a third example embodiment. A disease risk estimation device 80 includes acquisition means 81, an encoder 82, movement means 83, a predictor 84, and output means 85.

FIG. 14 is a flowchart of processing by the disease risk estimation device of the third example embodiment. The acquisition means 81 acquires a current age, a future age, and current attribute data other than the age (step S81). The encoder 82 projects the attribute data to a latent space according to a category of the age, and clusters obtained projection points into a plurality of clusters (step S82). The movement means 83 moves a projection point related to the current age to a position related to the future age in the latent space (step S83). The predictor 84 predicts a disease risk based on the position of the projection point on the latent space (step S84). The output means 85 outputs a prediction result of the disease risk (step S85).

According to the disease risk estimation device 80 of the third example embodiment, it is possible to estimate a future disease risk based on current data.

Some or all of the above example embodiments can also be described as the following Supplementary Notes, but are not limited to the following Supplementary Notes.

### (Supplementary Note 1)

A training device comprising:
an acquisition means for acquiring an age and attribute data other than the age;
an encoder that projects the attribute data to a latent space according to a category of the age and clusters obtained projection points into a plurality of clusters;
a predictor that predicts disease risks based on positions of the projection points on the latent space; and
an optimization means for optimizing the encoder and the predictor based on relationships between the projection points in the latent space and the plurality of clusters and a mutual relationship between the plurality of clusters.

### (Supplementary Note 2)

The training device according to supplementary note 1, wherein the optimization means optimizes the encoder and the predictor by using a loss function that decreases a loss as a distance between the projection point in the latent space and a center of gravity of the cluster to which the projection point belongs decreases and decreases the loss as a distance between the centers of gravity of the plurality of clusters increases.

### (Supplementary Note 3)

A Training Method Executed by a Computer, the Training Method Comprising:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

### (Supplementary Note 4)

A program for causing a computer to execute processing comprising:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

### (Supplementary Note 5)

A disease risk estimation device comprising:
an acquisition means for acquiring a current age, a future age, and current attribute data other than the age;
an encoder that projects the attribute data to a latent space according to a category of the age and clusters obtained projection points into a plurality of clusters;
a movement means for moving, in the latent space, a projection point related to the current age to a position related to the future age;
a predictor that predicts a disease risk based on the position of the projection point on the latent space; and
an output means for outputting a prediction result of the disease risk.

### (Supplementary Note 6)

The disease risk estimation device according to supplementary note 5, wherein the movement means moves the projection point related to the current age in such a way that a positional relationship between the projection point related to the current age in the latent space and a center of gravity of a cluster related to the current age matches a positional relationship between a projection point related to the future age in the latent space and a center of gravity of a cluster related to the future age.

### (Supplementary Note 7)

The disease risk estimation device according to supplementary note 5, wherein
the predictor predicts a current disease risk based on the projection point related to the current age, and predicts a future disease risk based on the projection point related to the future age, and
the output means outputs a comparison result between the current disease risk and the future disease risk.

### (Supplementary Note 8)

The disease risk estimation device according to supplementary note 5, wherein
the predictor predicts a current disease risk based on the projection point related to the current age, and predicts a future disease risk based on the projection point related to the future age, and
the output means outputs a comparison result between the current disease risk and the future disease risk.

### (Supplementary Note 9)

A disease risk estimation method executed by a computer, the disease risk estimation method comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.

### (Supplementary Note 10)

A program for causing a computer to execute processing comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Processor
Training device
21 Prototype encoder
22 Predictor
23, 24 Loss calculation unit
Loss integration unit
26 Optimization unit
28 Result output unit
100, 100x Disease risk estimation device

1. A training device comprising:
at least one first memory configured to store instructions; and
at least one first processor configured to execute the instructions to:
acquire an age and attribute data other than the age;
project, by an encoder, the attribute data to a latent space according to a category of the age and cluster obtained projection points into a plurality of clusters;
predict, by a predictor, disease risks based on positions of the projection points on the latent space; and
optimizes the encoder and the predictor based on relationships between the projection points in the latent space and the plurality of clusters and a mutual relationship between the plurality of clusters.

2. The training device according to claim 1, wherein the first processor optimizes the encoder and the predictor by using a loss function that decreases a loss as a distance between the projection point in the latent space and a center of gravity of the cluster to which the projection point belongs decreases and decreases the loss as a distance between the centers of gravity of the plurality of clusters increases.

3. A training method executed by a computer, the training method comprising:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

4. A non-transitory computer-readable recording medium storing a program for causing a computer to execute processing comprising:
acquiring an age and attribute data other than the age;
projecting, by using an encoder, the attribute data to a latent space according to a category of the age and clustering obtained projection points into a plurality of clusters;
predicting, by using a predictor, disease risks based on positions of the projection points on the latent space; and
optimizing the encoder and the predictor based on relationships between the projection points in the latent space and the plurality of clusters and a mutual relationship between the plurality of clusters.

5. A disease risk estimation device comprising:
at least one second memory configured to store instructions; and
at least one second processor configured to execute the instructions to:
acquire a current age, a future age, and current attribute data other than the age;
project, by an encoder, the attribute data to a latent space according to a category of the age and clusters obtained projection points into a plurality of clusters;
move, in the latent space, a projection point related to the current age to a position related to the future age;
predict, by a predictor, a disease risk based on the position of the projection point on the latent space; and
output a prediction result of the disease risk.

6. The disease risk estimation device according to claim 5, wherein the second processor moves the projection point related to the current age in such a way that a positional relationship between the projection point related to the current age in the latent space and a center of gravity of a cluster related to the current age matches a positional relationship between a projection point related to the future age in the latent space and a center of gravity of a cluster related to the future age.

7. The disease risk estimation device according to claim 5, wherein
the predictor predicts a current disease risk based on the projection point related to the current age, and predicts a future disease risk based on the projection point related to the future age, and
the output second processor outputs a comparison result between the current disease risk and the future disease risk.

8. The disease risk estimation device according to claim 5, wherein
the predictor predicts a disease risk of a subject based on the projection point related to the current age, and predicts an average disease risk based on a projection point related to an average value of the attribute data, and
the second processor outputs a comparison result between the disease risk of the subject and the average disease risk.

9. A disease risk estimation method executed by a computer, the disease risk estimation method comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.

10. A non-transitory computer-readable recording medium storing a program for causing a computer to execute processing comprising:
acquiring a current age, a future age, and current attribute data other than the age;
projecting the attribute data to a latent space according to a category of the age and clustering obtained projection points into a plurality of clusters;
moving, in the latent space, a projection point related to the current age to a position related to the future age;
predicting a disease risk based on the position of the projection point on the latent space; and
outputting a prediction result of the disease risk.
